(19) United States
(12) Patent Application Publication
(10) Pub. No.: US 2015/0320351 A1
(43) Pub. Date: Nov. 12, 2015

(54) Apparatus to measure the time and ability to process, react to, and synchronize complex visual effects relative to the detection of images in motion (based on the shannon-nyquist signal theory) with complex sounds or sound variations

(71) Applicant: Olivier L. Dehousse, New York, NY (US)

(72) Inventor: Olivier L. Dehousse, New York, NY (US)

(21) Appl. No.: 14/120,242

(22) Filed: May 12, 2014

(51) Int. Cl. A61B 5/00; A61B 5/16

(52) U.S. Cl. CPC A61B 5/4088; A61B 5/162

(57) ABSTRACT

In conjunction with the so-called “wagon wheel effect”, based on the Shannon-Nyquist signal processing theory, which our apparatus produces and measures under specific conditions, observed with one (left, right) or two eyes, when a disk reaches a speed , at which normal brain processes perceive as a rotation speed inversion, we create complex sounds or sound variations, which can be heard from one (left, right) or two ears, which we use to test in various scenarios individual's responses (potentially illustrating aspects of brain lateralization function) to support the establishment of a normative theory and measure specific populations (aging population, individuals suffering from mental illness such as Alzheimer, possibly in their early stage of development) against this norm.

### BACKGROUND OF THE INVENTION

1. Field of Invention

Our invention consists of an apparatus, which can reproduce under accurate measurable conditions, as defined per our embodiment, an experiment consisting of what is called the wagon wheel effect—at specific rotation speeds, a wagon wheel appears to rotate in a direction opposite to the real one—, and specific sounds or variation in sounds accurately detectable in time, which, in specific can be recreated at the specific time at which the appearing rotation inversion is perceived, and a method, consisting of collecting various measurements collected under various conditions to establish a norm, and allow for different populations, for instance aging populations or individuals suffering from nervous degenerative diseases, such as Alzheimer, possibly in their early stage of development, to be compared against this norm it is this maximum speed, which our apparatus is aimed to measure, which will be tested in coordination with a sound variation in various situations (left-right or both eyes, left-right or both ears). As such, our field of invention relates to a unique field of perception of images in motions and how it relates to hearing synchronization capabilities.

2. Related Background Arts

Our invention relies on a previous apparatus and method, which we have designed, to measure accurately a phenomena called the “wagon wheel inversion”, and which we introduced early on in a form of a patent. It is based on some fundamental observations or assumptions that we have made in various field of physiology, such as the lateralization of brain function and their origins. We give here also our original short views about potential origins for brain lateralization, which is also the reason for creating our invention. For many reasons which we won't explain in details here, animals have continued to live in heard. Such primitive social organization might be justified by natural genetic improvement of the species. And so do many fishes of small sizes, as they are shoaling or swimming in a school of fish. We could assume that brain lateralization could be a genetic predisposition leaving to an advantage for fishes leaving in banc. Indeed, when fishes are threatened by a predator, it is the combination of an image or a sequence of images (assuming fishes have likely very little image in motion processing capabilities due to the morphological positioning of their eyes and due to limited brain function) and sounds (giving them the indication for direction to follow to remain included in the school or move temporary away from it). (We could also f.i. further assume that fishes tend to try to gain a same position in a school). In simple brains, to have such function one next to the other might provide a genetic advantage for survival. It is at least the hypothesis that we are making here. Without being a medical doctor, we think that measurements showing correlation (or lack of thereof) between the hearing of complex sounds or sound variations and the detection of complex images in motion phenomena, as our invention (patent pending: Ser. No. 13/068,574) is designed to measure alone, might provide an interesting norm to assess strength (or lack of thereof) of brain lateralization in patient suffering for instance from Alzheimer.

New technology, such as fMRI (or functional magnetic resonance imaging using tracers to identify active area of the brain during specific tasks) makes possible to establish the dependencies between psychological processes, and their physical locations in the brain. Our experiment could also be reproduced in an MRI, allowing for instance to analyze the overlap of regions in activity, when the brain coordinates the processing of images in motion and the hearing complex sounds.

3. Cross-Reference

The provisional patent application (same owner) U.S. 61/465,772 is a related patent. It was introduced as a provisional patent, which foundations use the same principles as here.

Patent Ser. No. 13/068,574, with invention title “An apparatus to measure the speed at which, wheels in rotation present an appearing rotation speed inversion, the so called wagon wheel effect, with either one or two independent disks in rotation, with various spokelike patterns, and considering further characteristics specific to our design” is also considered previous art, foundation of this patent, and is pending at the time of this submission.

### BRIEF DESCRIPTION OF THE DRAWINGS

FIG. 1 in the accompanying drawing illustrating our embodiment of the invention is a schematic view of all the components along with one rotating disk, (One should note that our apparatus consists of two disks and therefore most of these components will need to be duplicated for our second disk).

FIG. 2 is a representation of the electrical circuit required for the functioning and control of our apparatus and the measurement of all the conditions that are here of interest in our case.

FIG. 3 shows a disk comprising various patterns, as an example of those that we will be using. It shows also an adjustable form, designed to further enhance the measurement of the effect, and provide a more accurate measurement of the rotation speed.

FIG. 4 shows some variations on the pattern which we will be using also, highlighting the use for multiple degrees of symmetry.

FIG. 5 shows a representation of our apparatus in 3D.

FIG. 6 indicates that either one of the rotating disks can be used or both rotating disks can be used independently and at the same time in our apparatus.

FIG. 7 shows our device used to create complex sounds or sound variation, which can be triggered at the time of the appearance of the rotation inversion.

### DETAILED DESCRIPTION OF THE EMBODIMENT

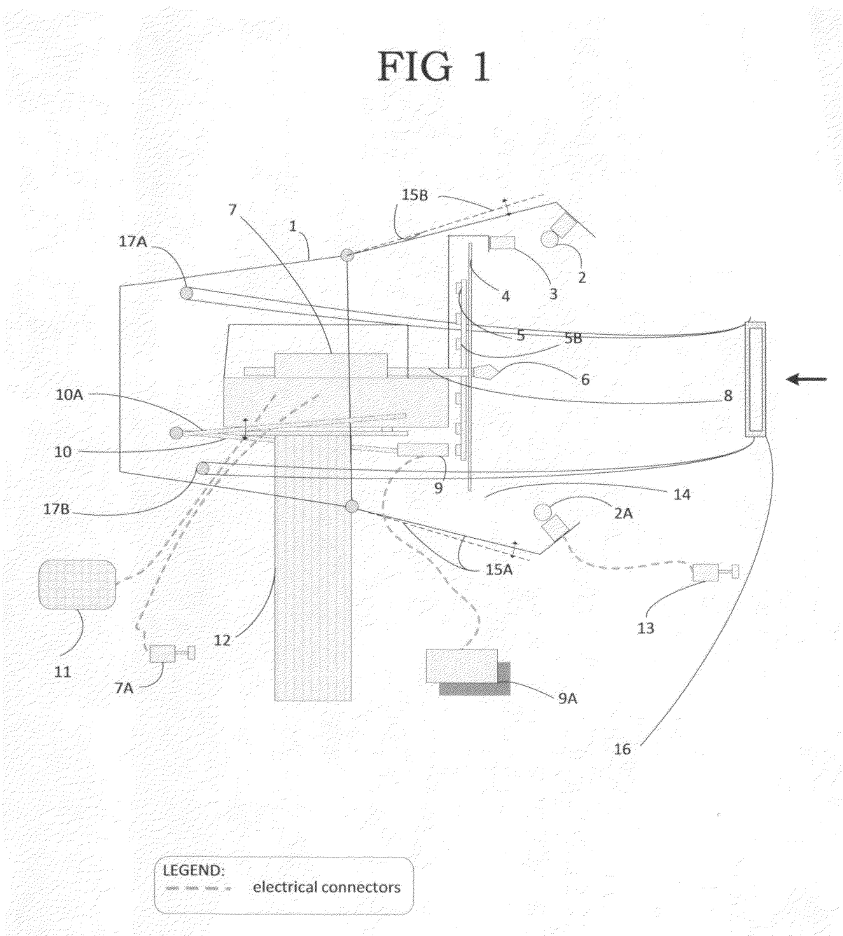

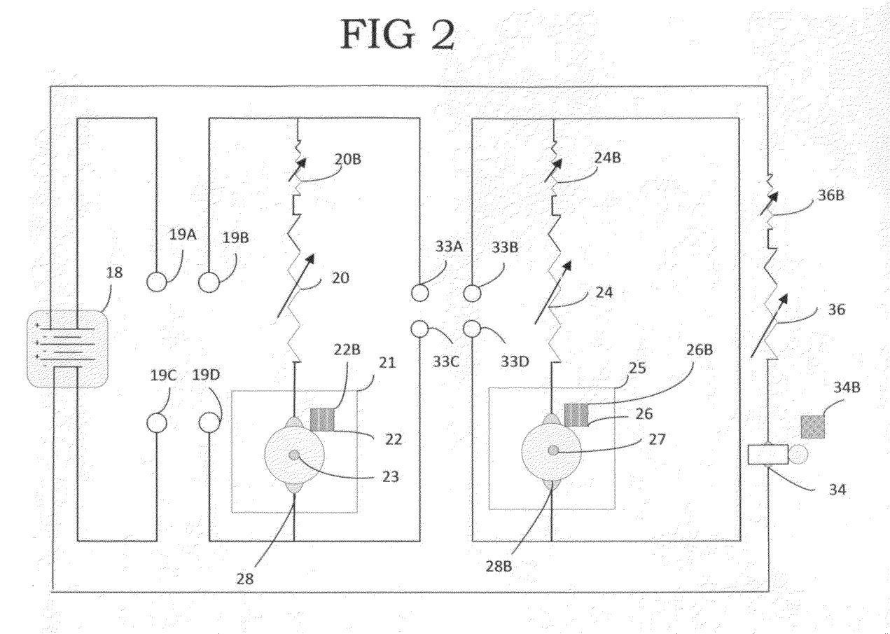

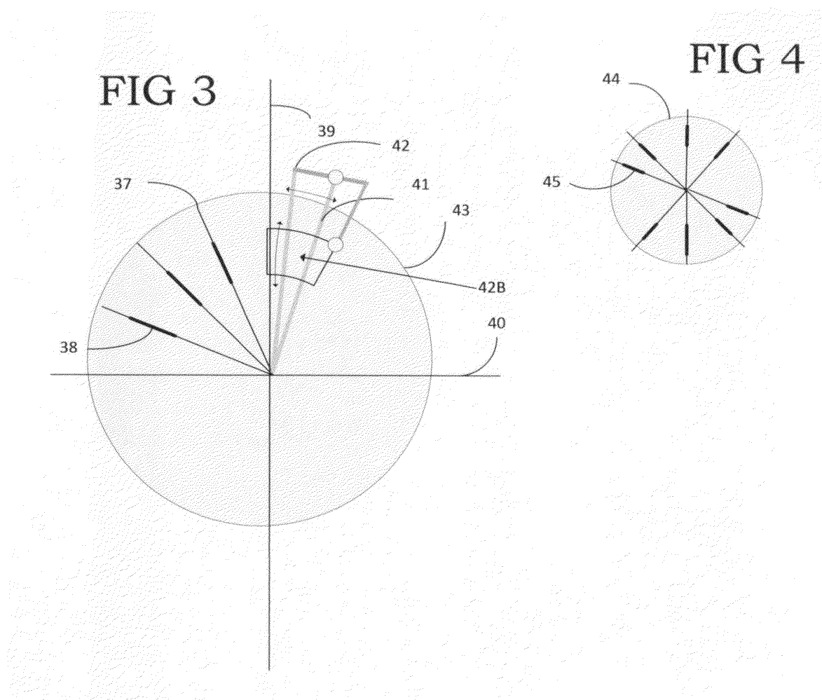

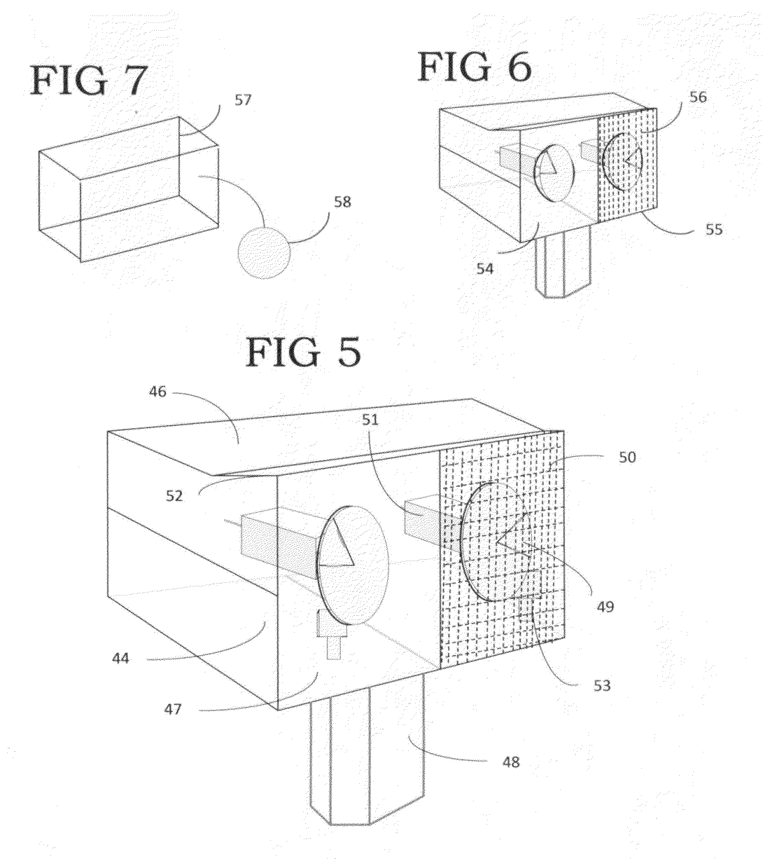

FIG. 5 and FIG. 6 represent a simplified 3D view of our apparatus, which includes two disks capable of rotating independently across a large range of measurable speeds. An observer is maintained at a specific distance from the disks in using an open form 16 attached to an extension, itself attached to the housing of our machine at points 17A and 17B on the FIG. 1. The combination of the open form and the extension are used to maintain an observer directly in front of the two rotating disks and at a specific distance from them. FIG. 5 and FIG. 6 show a simplified 3D representation of our apparatus with the two disks as seen by an observer. A screen 41 can be placed in front of either rotating disk to prevent from being seen by an observer, and can further be moved to the left side 42A or the right 42B at any time. The housing 1 is comprised of an adjustable stand 12, which allows for a comfortable positioning of the apparatus and the observer. Furthermore, the position of the disks will always be centered and face the observer's face is in perfect horizontal alignment with an observer's view as showed by the arrow on FIG. 1. This position can also be adjusted such as one side of each shaft forms a small angle with an horizontal positioning. A simple mechanism is provided to elevate a side of the disks' shafts and create this angle 10 and 10A. Two electrical motors 7, side by side, included in the housing, are respectively connected to a shaft 8, which via a simple compound gear system, rotates with their respective motors. At the end of each of these shafts, there is a circular support 4 used to attach various disks, variable in size, in color, in spokelike patterns, in thickness and in their capacity to reflect light—matt or glossy finish. The type of motors preferred is DC as this type of electrical motor offers a better and easier control over speed by the use of a potentiometer 9 to regulate the voltage at their armature. The external source of DC electrical power is also represented in 11.

The housing of our apparatus is capable to adjust to different sizes of disks in using moveable superior 15A and inferior 15B walls. Each shaft 8 is terminated by a knob 6 to attach the various disks to the front side of the circular support 4 itself attached to the motor. On the opposite side of the rotating circular support, multiple small reflective elements 5 can be added along the circumference of each disks at regular angles to reflect light (infrared preferred) emitted by the tachometer source 8. Each source (respectively for each disk) is capable to count the number of refection of the light per unit of time as the disk is in rotation, and as such to provide an accurate measurement of the disk rotation angle per unit of time at any time. The accuracy of the speed measured can be increased by increasing the number of these reflective elements 5 capable to reflect the infrared light. In our simplified embodiment, the mechanism provided to measure the speed, the tachometer, comprising of a source 8 and the reflecting elements 5, has its own power generator (It is using internal battery). A screen connected to the tachometer 8A is included to show an accurate measurement of the rotation speed when the rotation inversion—the so-called wagon wheel effect—takes place for an observer. To measure variable conditions in which the effect takes place, a system comprised of a control over light intensity is also included, consisting of sources of light 2 and 2A and potentiometers 13 controlling their light intensity. The light intensity can be measured with a light meter 3, providing a measurement of the lightning conditions in which the experiment takes place, which needs to be uniform over the two entire disks surface.

The source of DC current 18 alimenting the two armatures 28 and 28B of the two DC electrical motors can be inverted with a switch connecting the contacts 19AD and 19BC to become 19AC and 19BD. Another similar switch placed between the two motors connecting the contacts 33AD and 33BC can become 33AB and 33CD allows to change the rotation direction of the two motors and the motors' shafts, respectively showed in 23 and 27. The two motors 21 and 25 are confined in a single housing as presented in 34 (FIG. 5). Two series of potentiometers different in size 20 and 20B and 24 and 24B allow for the gradual adjustment of the rotation speed and its accurate measurement when the wagon wheel effects takes place. Two independent tachometers 22 and 26 connected to a screen are respectively place at the extremity of each circular support to measure each rotation speed independently. Another circuit enables to control the light 34, intensity and lightning environment with dual potentiometers 36 and 36B. These lightning conditions can also be measured any time with the appropriate light meter 34B.

The rotating disks designed to produce the wagon wheel effect present spokelike patterns 43 and 44 with various levels of symmetry, which is important in the context of the use of our apparatus, (a higher levels of symmetry will produce the wagon wheel effect at a lower rotation speed for a same observer). The spokes designed on the disks will present different thickness 37 and 38 sometimes even in specific areas of the spoke. The spokes 28 designed on a disk, as well as these thicker areas can also be drawn in different colors (because different types of eye cells react differently to different colors) and in a material capable to reflect the light differently (matt or glossy finish). A variable form 42 which goal is for an observer to focus on a specific band region (area of interest) of the rotating disk is also part of our apparatus. This variable form is attached to superpose the disk and remain static during the experiment while the disk is rotating. As the area of interest become smaller with the adjustment of the triangular surface 42, the appearing speed rotation inversion becomes restricted to the area of interest and adds accuracy to the measurement. As such the reduced area of interest can further limit the view of an observer in focusing on a specific band on each disk, in modifying the band's distant to the center of the disk, itself directly proportional to the speed, as showed by the arrow 42B. Finally, this form has one edge centered around the shaft and two vertices of equal size which can be moved apart or brought back together to increase or decrease the rectangular area.

A simplified version of our apparatus in 3D is in FIG. 5 and FIG. 6 and shows the housing 46, the superior moving wall 52 allowing for large disks to be inserted in the machine, and an adjustable stand 48. FIG. 5 shows also the two disks and their respective electrical motors, to which there are connected via a shaft. Our apparatus allows for one disk and two disks to rotate independently 54 and 55. An opaque screen 56 can be used to block the view of either the left or the right rotating disk at any time.

FIG. 7 shows the symbolic embodiment of a device capable of emitting on demand complex sounds corresponding to the best hearing assessment which can be detected in the shortest time period. In other words, the device will create a sound or a variation in a sound, such as a pitch for instance, at a specific time that can be identified. In specific, this device will be responsible for emitting a sound at the time (based on the speed of one or two disks) when the so called “wagon wheel inversion” is detected in various situations, permitting to assess the response between visual and hearing capabilities in a subject. It is a required embodiment for the method used as experiment. As such the 57 embodiment include some control and a 58 hearing device, which can be used on a left or a right ear too listen to the sounds or sound variations as described above.

### SUMMARY OF THE INVENTION

In accordance with our present invention, a machine for accurately measuring the rotation speed of one disk for one eye (left or right) or two eyes at which an observer perceives a rotation speed inversion (according to the so called “wagon wheel effect” based on the Shannon-Nyquist signal theory), and which is capable to create complex sounds or sound variations at specific times, in specific when an appearing inversion in rotation is perceived, which can be used by one ear (left or right) or both ears. Our long-term goal is to provide an apparatus which can be used to establish a normative theory about coordination between the wagon wheel effect and sounds, in such a way that complex visual processing of images in motion can be measured in time against complex sound perceptions left-left and right-right or left-right (we mean by that left eye-right ear and so), potentially measuring for the first time some effect of brain lateralization (or lack of thereof) or damages to brain lateralization function in patient brain suffering from degenerative diseases such as Alzheimer, which today we believe, does not exist, and to measure specific populations (aging population, individuals suffering from mental illness such as Alzheimer, possibly in their early stage of development) against a potential norm.

I claim:

1. An apparatus for producing and measuring accurately, in specific conditions, the speed at which the so-called “wagon wheel effect” is produced—at specific speed(s), a wagon wheel appears to rotate in a direction opposite to the real one—with two independent rotating disks, presenting various spokelike patterns, under specific conditions of light and colors, comprising:
a. A housing comprising:
i. Two disks capable to rotate independently across a wide range of speeds which can be accurately controlled, adjustable in sizes, in colors, in material reflecting light intensity, presenting various spokelike patterns.
ii. An embedded tachometer allowing the measurement of the disks' speed with accuracy at any time, and allowing the reading of such speeds at any time.
iii. An embedded system producing uniform light, variable in intensity, across the disks at any time and a light meter accurately measuring the light intensity at any time.
iv. An embedded mechanism to consistently position our apparatus against an observer.

2. The machine of claim 1 further comprising an accurate mechanism of speed control used for the two rotating disks, capable to accurately control the two disks independently in speed and in direction, and to measure it wherein:
a. Potentiometers of various sizes and sensitivity can accurately control the speed in controlling the voltage on the armature of electrical motors.
b. Switches allow the inversion of the respective difference in voltages on the armature of the electrical motors, independently of the source of current used.
c. Reflective elements, part of the embedded tachometer system, placed at regular angles along each of the circular support of each of the disks, which count can be increased and therefore increase the sensitivity of the speed measurement and give an accurate measurement of the rotation speed in angle (degrees) per second.

3. The machine of claim 1 further comprising a sensitive mechanism consisting of potentiometers producing a uniform lightning environment, uniform on and in the surrounding of the two disks variable in size, and allowing with the support of a light meter to measure the light intensity accurately at any time.

4. The machine of claim 1 further comprising of an extension allowing for the specific positioning of an observer in front of one or the two disks in rotation and at specific consistent distance from these disks, which further sets the distance separating our apparatus from an observer.

5. The machine of claim 1 further comprising disks presenting spokelike patterns, with spokes of various thicknesses, various levels of symmetry, various sizes, various colors and using various materials capable to reflect the light intensity differently.

6. The machine of claim 1 further comprising an adjustable area, in turn defining a specific area of focus on each rotating disks, which surface can increase or decrease in moving apart or bringing together the two vertices, and an upper and lower limit restraining the view of the adjustable disk area to a specific band to further refine the speed measurement.

7. A device capable to emitting sounds or sound variations, which can be accurately detected in time by one (left or right) or two ears, and triggered at the time (based on the speed of one or two disks) when a inversion in the rotation of one or two disks is perceived.

8. A method consisting of measuring accurately in time all characteristics presented per our design to create a norm and to measure various populations against such norm. Such populations should comprise aging populations, populations under the effect of short or long term conditions such as induced by alcohol consumption, or suffering from diseases, such as Alzheimer or other degenerative nervous diseases, possibly in their respective early stages of development.
